(19) United States
(12) Patent Application Publication
(10) Pub. No.: US 2014/0170126 A1
(43) Pub. Date: Jun. 19, 2014

(54) PROBIOTIC FOR ADMINISTRATION TO HEALTHY YOUNG MAMMALS DURING THE WEANING PERIOD FOR IMPROVING TOLERANCE TO NEWLY INTRODUCED FOOD STUFFS

(71) Applicants: Swantje Duncker, Lausanne (CH); Marie Lewis, Cheddar Somerset (GB); Annick Mercenier, Bussigny (CH); Anurag Singh, Ecublens (CH); Michael Bailey, Bristol Somerset (GB)

(72) Inventors: Swantje Duncker, Lausanne (CH); Marie Lewis, Cheddar Somerset (GB); Annick Mercenier, Bussigny (CH); Anurag Singh, Ecublens (CH); Michael Bailey, Bristol Somerset (GB)

(73) Assignee: NESTEC S.A., Vevey (CH)

(21) Appl. No.: 14/232,506

(22) PCT Filed: Jul. 11, 2012
(86) PCT No.: PCT/EP2012/063553

§ 371 Date: Jan. 13, 2014

(30) Foreign Application Priority Data
Jul. 12, 2011 (EP) 11173567.6

(51) Int. Cl. A61K 35/74; A23L 1/30

(52) U.S. Cl. CPC A61K 35/747; A23L 1/3014; A61K 35/745
USPC 424/93.45; 424/93.4

(57) ABSTRACT

The current invention is based upon administration of a probiotic, B. Lactis NCC2818 to healthy young mammals during the critical weaning period (in infants this period is usually from about 3 months to about 12, 18 or 24 months old), so as to accelerate the young mammal's adaptation to new food. The effectiveness of the invention is evidenced herein by morphological and immunological changes observed in a piglet animal model of weaning. Thus, administration of the probiotic according to the invention had a prophylactic effect, preventing the severe discomfort and pathological states associated with the introduction to novel foods during the weaning period.

### FIELD OF THE INVENTION

This invention relates to improving tolerance in young mammals, especially human infants, to newly introduced foods during the weaning period, by administering a probiotic or mixture of probiotics.

### BACKGROUND TO THE INVENTION

Post-Natal Maturation of the Intestinal Immune System

Infants as well as other young mammals are born with a functional but naïve (non-educated) intestinal immune system. Full immune competence is gradually achieved after birth and can only be accomplished through education of the immune system with progressive encounter of external stimuli, such as ingested proteins and/or the intestinal microbiota. This gradual immune maturation eventually results in the competence to distinguish between harmful and harmless stimuli and mounting of appropriate immune responses (meaning inflammation upon encounter of pathogens, and tolerance when food components and commensal bacteria are encountered). Thus, infancy is an unstable time for the immune system with dichotomous outcome possibilities leading either to tolerance and protective immunity or to pathological allergic immune responses (Cummins and Thompson; 1997; Immunology and Cell Biology; 75,419-29).

During the post-natal maturation of the intestinal immune system, mothers' milk ensures immune protection and compensates for the lack of immune capacity in the intestine. However, exclusive breast milk-feeding can only sustain adequate nutritional support for a limited time after birth, i.e. 4 to 6 months in human infants. After this period, other foodstuffs are progressively introduced into the diet to meet the nutritional needs of the infant, and the dependence on milk or formula to provide all the nutrients is thereby reduced. This process is commonly referred to as weaning. In human infants, weaning onto complementary foods occurs gradually from 3 months to 12 months of age. However, the age at which complementary food are introduced may vary according to geographic location and cultural differences (Aggett, P. J., Research priorities in complementary feeding: International Paediatric Association (IPA) and European Society of Paediatric Gastroenterology, Hepatology, and Nutrition (ESPGHAN) workshop. Pediatrics 2000; 106:1271). Other mammals, like dogs and cats, wean themselves gradually from mother's milk, starting to eat complementary food at 3-4 weeks and becoming independent of milk at 8-10 weeks old.

Maturation of the gastrointestinal tract in infants and young mammals comprises a number of physiological mechanisms that take place in infancy, and that all contribute to the evolution of an immature gastrointestinal system into a mature adult one. One of the key steps involved is adaptation to new food, which mainly takes place during weaning. Therefore, adaptation to new foods at weaning is seen as an important part of gastrointestinal maturation.

The Immune System and Intestinal Physiology Undergo Modifications Around Weaning

The intestinal immune system of the healthy young mammal is activated around the weaning period. This activation includes humoral and cellular mechanisms and is a response to the high amount of newly encountered antigens as a result of the change in food sources (milk to solids). It has been shown that this initial immune activation at weaning, in response exposure to new food in mammals, is transient. In rats, for example, weaning is associated with an increased cell number in the mesenteric lymph nodes (MLN), an increased number of jejunal lymphocytes, and mast cell degranulation. Human infants show expansion of duodenal mast cells and an increase in intraepithelial lymphocytes (Thompson, F. M., Mayrhofer G, Cummins A. G., Dependence of epithelial growth of the small intestine on T-cell activation during weaning in the rat, Gastroenterology 1996; 111:37-44). It has also been shown in mice that the number of spontaneous cytokine secreting cells increases transiently during weaning (Vazquez, E., Gil, A., Garcia-Olivares, E., Rueda, R., Weaning induces an increase in the number of specific cytokine-secreting intestinal lymphocytes in mice, Cytokine 2000; 12:1267-70).

Transient immune activation around weaning is believed necessary for the education of the intestinal immune system, subsequently rendering the growing infant tolerant towards harmless stimuli (e.g. food, commensal bacteria). It is common understanding that one of the ways to physiologically achieve intestinal tolerance is by downregulation of initial local immune responses against a new stimulus.

Weaning not only impacts the intestinal immune system, but also initiates substantial, food-induced changes in the metabolism and the morphology in the intestine. Intestinal morphology is usually accessed by morphometry of the villi (villus length or villus area) and crypts (crypt length and fission). Human infants, for example, show an increase in crypt fissions at an age of 6-12 months, as well as an increase in crypt length between 12 and 24 months and a decrease in villus area around weaning (Cummins, A. G., Catto-Smith A. G., Cameron, D. J. et al., Crypt fission peaks early during infancy and crypt hyperplasia broadly peaks during infancy and childhood in the small intestine of humans, J. Pediatr. Gastroenterol. Nutr., 2008; 47:153-7). As with the immune system most of these morphological changes are transient and reach a balance in children at an age of about 4 years to resemble the adult situation.

Unfortunately, the activated immune status of the healthy young mammal at weaning—necessary for appropriate immune responses during later life—, as well as the morphological changes in the intestine, make the young mammal more vulnerable to stresses it may encounter at the same time. This vulnerability can result in weaning associated complications, like the highly common, chronic nonspecific childhood diarrhea (Kleinman, R. E., Chronic nonspecific diarrhea of childhood, Nestlé Nutr. Workshop Ser. Pediatr. Program, 2005; 56:73-9) or an inadequate immune system response to food proteins, namely, food allergy, hypersensitivity and food protein induced enterocolitis (FPIES) (Nowak-Wegrzyn, A., Muraro, A., Food protein-induced enterocolitis syndrome, Curr. Opin. Allergy Clin. Immunol., 2009; 9:371-7). Of course, the weaning-associated pathological states mentioned above are a source of discomfort to the young mammal.

Furthermore, with the increased intake of complementary foods, the infant is exposed to a higher number of potential pathogenic microorganisms (Sheth, M., Dwivedi, R., Complementary foods associated diarrhea, Indian J. Pediatr., 2006; 73:61-4) thereby increasing the risk of infection. During weaning, while food intake is increased, the intake of breast milk is progressively decreased. Thus, there is less consumption of the immune protective compounds found in human milk at a time when these compounds are most needed, and the immune system of the young mammal is not yet capable to fully provide these factors.

Complications around weaning are especially detrimental, because the shaping of the immune system at this time can have a long lasting impact on how immune challenges are dealt with later in life. This has been shown, for example, in food allergy, type-1 diabetes and celiac disease.

Gastrointestinal Microbiota and Weaning

One of the main influences driving the development and maturation of the immune system is early colonization of the intestine with microorganisms. It has been shown that animals, reared under germfree conditions, have a severely under-developed intestinal immune system which can be rescued by introduction of commensal bacteria and/or probiotics. It has also been demonstrated that, during the first months of life, mammals undergo considerable fluctuation in the composition of their intestinal microbiota. Whereas Bifidobacteria dominate during breast feeding, the microbiota becomes more complex with the introduction of complementary foods. It is then dominated by Bacteroitedes, Enterococci, and anaerobic cocci after weaning.

Since the process of weaning is associated with a major shift in the nature of the intestinal microbial community, this period represents a window for intervention, for example, with probiotics. Furthermore, modification of the developing microbiota by intervention with probiotics during weaning may have a more pronounced impact on the subsequent function of the immune system than administration of probiotics to adults.

Thus, it is not surprising that weaning is a critical and physiologically challenging time during normal development, and is considered as a stress for the young mammal. Accordingly, there is a need to help the young mammal through the critical weaning period with the least discomfort possible, while ensuring he consumes adequate food to satisfy the nutritional needs. There is a need to provide a therapeutic treatment that can prevent weaning associated conditions, in particular, those mentioned in the paragraph above including chronic nonspecific childhood diarrhea and food protein induced enterocolitis syndrome (FPIES). There is need to provide a prophylactic therapeutic treatment to prevent or attenuate the symptoms of weaning associated conditions.

Additionally, there is a need to facilitate and accelerate the adaptation of the gut of the young mammal to new foods encountered during the weaning period.

There is a need to induce or support tolerance towards newly introduced foods during the weaning period.

There is a need to prevent and treat intestinal discomfort felt by the young mammal associated with weaning. This discomfort may be minor, and not indicative of a particular pathological state. Alternatively, the discomfort can be severe, giving rise to pain, and prolonged crying in the infant. This severe discomfort may be associated with severe pathological conditions.

### SUMMARY OF THE INVENTION

The current invention responds to the needs described above. The invention is based upon administration of a probiotic to healthy young mammals during the critical weaning period (in infants this period is usually from about 3 months to about 12, 18 or 24 months old), so as to accelerate the young mammal's adaptation to new food. The effectiveness of the invention is evidenced herein by morphological and immunological changes observed in a piglet animal model of weaning, in which intestinal mucosal villus physiology, antigen specific IgG₁ and IgG₂ levels in serum, and the number and type of B cell follicles in MLN (mesenteric lymph node) cells were measured.

Thus administration of the probiotic results in an enhancement of the transient increase in the humoral immune response, in particular, in immunoglobulin class G production, upon exposure to newly introduced foods. The increase occurs more rapidly and/or to a greater extent, compared to that occurring in young mammals not receiving the probiotic.

Thus administration of the probiotic, during weaning, results in an increase of more than 15% in the height and/or area of the intestinal mucosal villi compared to that of young mammals not receiving the probiotic.

The invention concerns the prevention of pathological states associated with weaning such as chronic nonspecific childhood diarrhea, an inadequate immune system response to food proteins, namely, food allergy, hypersensitivity and FPIES. Thus, symptoms associated with lack of tolerance to newly introduced food during weaning are prevented, or reduced at weaning and later in life. At the same time, the intervention allows a normal immune adaptation of the young mammal. Thus, the period during which the young mammal has an increased vulnerability due to weaning is reduced.

Thus, administration of the probiotic according to the invention had a prophylactic effect, preventing the severe discomfort and pathological states associated with the introduction to novel foods during the weaning period.

The invention also aims to prevent minor intestinal discomfort associated with weaning.

The probiotic administered is preferably Bifidobacterium animalis subsp. lactis (B. Lactis), strain B. lactis CNCM-I-3446, also known as B. lactis NCC2818. The probiotic may be live or have been inactivated to render it non-replicating. The daily dose that may be used is of from 10² to 1×10¹¹, preferably 1×10⁶ to 1×10⁹ cfu (cfu=colony forming unit) or equivalent of cfu in case of non-replicating microorganisms.

The probiotic may be administered in its pure form, or diluted in water, or in a composition suitable for administration to young mammals. The latter composition may comprise other additional probiotics, preferably selected from Bifidobacterium longum BB536 (ATCC BAA-999); Lactobacillus rhamnosus (CGMCC 1.3724); Lactobacilus reuteri (DSM 17938) or mixtures thereof. The composition may also comprise prebiotics such as inulin, fructooligosaccharide (FOS), short-chain fructooligosaccharide (short chain FOS), galacto-oligosaccharide (GOS), xylooligosaccharide (XOS), arabinoxylan-oligosaccharides (AXOS), glangliosides, partially hydrolysed guar gum, acacia gum, soybean-gum. The composition may also comprise non-prebiotics like Lactowolfberry, wolfberry extracts or mixtures thereof.

The composition may be an infant formula, a follow-on formula, or growing-up milk, a baby cereal or yoghurt, a baby meal, pudding or cheese, a dairy or fruit drink, a smoothy, a snack or biscuit or other bakery item. The composition may be in the form of a shelf-stable or freeze-dried product, or be produced by extrusion, aseptic process or retort.

### BRIEF DESCRIPTION OF THE DRAWINGS

FIG. 1: Feeding Schemes

A: Feeding Scheme I: Piglets were weaned from mother's milk onto solid food (Soya or OVA (egg) based protein, respectively) and one group was supplemented with NCC2818. All groups were changed to fishmeal from 49 days until termination of the experiment at 77 days. n=6.

B: Feeding Scheme II Piglets were fed formula from 24 h of age, with or without NCC2818. Half of the piglets of each group were then either weaned onto an egg protein based diet, or not weaned at all. The experiment was terminated at 25 days of age. n=6.

FIG. 2: Serum IgG Response to Fed Soya

Change of soya-specific IgG₁ (A) and IgG₂ (B) in serum of soya-fed piglets, either supplemented (Soya+NCC2818), or non-supplemented with NCC2818 (Soya diet), or non-supplemented, egg-fed piglets (Egg diet). Error bars =SEM (n=14). Results are expressed as the change of antibody levels to soya protein after intervention, compared to that before intervention (the —fold change in antibody).

FIG. 3: Histomorphometry of the Intestinal Mucosa (Distal Small)

Villus height of piglets fed with, or without NCC2818, from 24 h onwards. Pigs were either weaned onto solid food (Egg diet, Egg diet+NCC2818) at day 21, or kept on piglet formula (Formula). Histomorphometry analysis was carried out after termination of the experiment at day 25. Results are presented as mean log₁₀ mm±Standard Error (SE).

FIG. 4: Fluorescence Immunohistology of B-Cell Follicles in Mesenteric Lymph Node (MLN) Cells

Total number of follicles (A), expressing IgA and IgM in extrafollicular cells (B), and number of IgA or IgM positive follicles (C) of soya-fed piglets either supplemented or non-supplemented with B. lactis NCC2818 when weaning started at day 21. Error bars=SEM (n=6).

### DETAILED DESCRIPTION OF THE INVENTION

Definitions

In this specification, the following terms have the following meanings:

“Weaning period” is the period during which young mammals are adapting from pure liquid milk based nutrition to semi-solid or solid foods, and adapting from a quasi-unique food type (generally, in the case of infants, mother's milk or infant formula) to a variety of foods.

“Tolerance” means an active state of hypo-responsivness to food.

“Probiotic” means microbial cell preparations or components of microbial cells with a beneficial effect on the health or well-being of the host. (Salminen, S., Ouwehand, A. Benno, Y. et al., Probiotics: how should they be defined, Trends Food Sci. Technol. (1999): 10 107-10). The definition of probiotic is generally admitted and in line with the WHO definition. The probiotic can comprise a unique strain of micro-organism, a mix of various strains and/or a mix of various bacterial species and genera. In case of mixtures, the singular term “probiotic” can still be used to designate the probiotic mixture or preparation. For the purpose of the present invention, micro-organisms of the genus Bifidobacterium are considered as probiotics.

“Prebiotic” generally means a non digestible food ingredient that beneficially affects the host by selectively stimulating the growth and/or activity of micro-organisms present in the gut of the host, and thus attempts to improve host health.

Bifidobacterium animalis subsp. lactis (B. lactis) strain NCC2818 (Nestlé Culture collection) is the B. lactis deposited under the international identification reference CNCM-I-3446 (Collection Nationale de Cultures de Microorganismes at Institute Pasteur, Paris, France). B. lactis NCC2818 is used throughout the text. The CNCM identification refers to the Collection Nationale de Cultures de Microorganismes at Institut Pasteur, 22 rue du docteur Roux, 75724 Paris, France.

The invention concerns the administration of a probiotic, in particular, B. lactis NCC2818 (B. Lactis CNCM-I-3446) to healthy young mammals, during the weaning period, i.e. when the young mammal starts to consume non-milk food and depends less and less on milk for his nutritional requirements. In human infants, this period occurs usually when the infant is approximately 3 months to 12 months old, although the period may extend to 18, 24 or even up to 36 months old. Infants generally continue to regularly encounter new foods up until this latter age, and even older.

Details of the mode of administration of the probiotic are given in the following paragraphs.

As is demonstrated by the experimental data of Example 1, administration of B. lactis NCC2818 to piglets at weaning can have marked effects on the structure and functions of the gut-associated mucosal immune system. The probiotic administration according to the invention accelerates the adaptation of the young mammal to newly introduced foods and improves the young mammal's tolerance to newly introduced foods. Thus, the intervention provides a method to support the infant's immune adaptation during the challenging time of weaning. All infants may benefit from the present invention, including those at risk of developing atopic diseases because of their family history.

Doses of Probiotic

The daily doses of B. lactis NCC2818 administered to the young mammal are from 1×10⁶ to 1×10¹¹ cfu, preferably 1×10⁶ to 1×10⁹ cfu (cfu=colony forming unit).

B. lactis NCC2818 may be present in a composition administered to the young mammal in a wide range of percentages provided that it delivers the positive effect described. Thus the amount of probiotic present per gram of dry composition for administration may vary as long as the daily doses described above are respected. However, preferably, the B. lactis NCC2818 is present in the composition in an amount equivalent to between 1×10² and 1×10¹¹ cfu/g of dry composition, preferably 1×10⁴ to 1×10⁹ cfu/g of dry composition. This includes the possibilities that the bacteria are live, inactivated or dead or even present as fragments such as DNA or cell wall materials. Methods known in the art may be employed to render the probiotic non-replicating. Thus, the quantity of bacteria which the formula contains is expressed in terms of the colony forming ability of that quantity of bacteria as if all the bacteria were live irrespective of whether they are, in fact, live, inactivated or dead, fragmented or a mixture of any or all of these states.

Method of Administration

The B. lactis NCC2818 can be administered orally to the young mammal; this may be pure or diluted in water or mother's milk for example, as a food supplement or as an ingredient in an infant milk formula. Such a formula may be an infant “starter formula” if probiotic administration starts before the infant is 6 months old, or a “follow-on formula” if the infant is older than 6 months. An example of such starter formula is given in Example 2. The formula may also be a hypoallergenic (HA) formula in which the cow milk proteins are hydrolysed.

If the young mammal is between 12 and 24 months old the probiotic may be administered in a growing-up milk, cereal or yoghurt, baby meal, pudding or cheese, dairy and fruit drink, smoothy, snack or biscuit or other bakery item. An example of such growing-up milk is given in Example 3. The composition may be in the form of a shelf stable or freeze dried product, or may have been produced by extrusion, an aseptic process or retort.

Administration with Other Compounds

The B. lactis NCC2818 may be administered with one or more additional probiotics. These probiotics are preferably selected from Bifidobacterium longum BB536 (ATCC BAA-999); Lactobacillus rhamnosus (CGMCC 1.3724); Lactobacilus reuteri (DSM 17938) or mixtures thereof.

The B. lactis NCC2818 can be administered alone (pure or diluted in water or milk, including breast milk for example) or in a mixture with other compounds (such as dietary supplements, nutritional supplements, medicines, carriers, flavours, digestible or non-digestible ingredients). Vitamins and minerals are examples of typical dietary supplements. In a preferred embodiment, the composition is administered together with other compounds that enhance the described effect on the immunity of the progeny. Such synergistic compounds may be carriers or a matrix that facilitates the B. lactis NCC2818 delivery to the intestinal tract of the young mammal. Such compounds can be other active compounds that synergistically, or separately, influence the immune response of the infant and/or potentiate the effect of the probiotic. An example of such synergistic compounds is maltodextrin. One effect of maltodextrin is to provide a carrier for the probiotic, enhancing its effect, and to prevent aggregation.

Other examples include known prebiotic compounds such as carbohydrate compounds selected from the group consisting of inulin, fructooligosaccharide (FOS), short-chain fructooligosaccharide (short chain FOS), galactooligosaccharide (GOS), xylooligosaccharide (XOS), arabinoxylan oligosaccharides (AXOS), glangliosides, partially hydrolysed guar gum (PHGG) acacia gum, soybean-gum, apple extract, and non-prebiotic compounds like Lactowolfberry, wolfberry extracts or mixture thereof. Other carbohydrates may be present, such as a second carbohydrate that may act in synergy with the first carbohydrate. The carbohydrate or carbohydrates may be present at about 1 g to 20 g or 1% to 80% or 20% to 60% in the daily doses of the composition. Alternatively, the carbohydrates are present at 10% to 80% of the dry composition.

The daily doses of carbohydrates, and all other compounds administered with the B. lactis NCC2818 should always comply with the published safety guidelines and regulatory requirements. This is particularly important with respect to the administration to young infants, under one year old.

In one embodiment, a nutritional composition preferably comprises a source of protein. Dietary protein is preferred as a source of protein. The dietary protein may be any suitable dietary protein, for example animal proteins (such as milk proteins or meat proteins), vegetable proteins (such as soy proteins, wheat proteins, rice proteins or pea proteins), a mixture of free amino acids, or a combination thereof. Milk proteins such as casein and whey proteins are particularly preferred.

The composition may also comprise a source of carbohydrates and/or a source of fat.

If the composition of the invention is a nutritional composition and includes a fat source, the fat source preferably provides about 5% to about 55% of the energy of the nutritional composition; for example about 20% to about 50% of the energy.

Lipid making up the fat source may be any suitable fat or fat mixture. Vegetable fat is particularly suitable, for example soy oil, palm oil, coconut oil, safflower oil, sunflower oil, corn oil, canola oil, lecithin and the like. Animal fat such as milk fat may also be added if desired.

An additional source of carbohydrate may be added to the nutritional composition. It preferably provides about 40% to about 80% of the energy of the nutritional composition. Any suitable carbohydrate may be used, for example sucrose, lactose, glucose, fructose, corn syrup solids, maltodextrin, or a mixture thereof. Additional dietary fibre may also be added if desired. If added, it preferably comprises up to about 5% of the energy of the nutritional composition. The dietary fibre may be from any suitable origin, including for example soy, pea, oat, pectin, guar gum, acacia gum, fructooligosaccharide or a mixture thereof. Suitable vitamins and minerals may be included in the nutritional composition in an amount to meet the appropriate guidelines.

One or more essential long chain fatty acids (LC-PUFAs) may be included in the composition. Examples of LC-PUFAs that may be added are docosahexaenoic acid (DHA) and arachidonic acid (AA). The LC-PUFAs may be added at concentrations so that they constitute greater than 0.01% of the fatty acids present in the composition.

One or more food grade emulsifiers may be included in the nutritional composition if desired; for example diacetyl tartaric acid esters of mono- and di-glycerides, lecithin and mono- or di-glycerides or a mixture thereof. Similarly suitable salts and/or stabilisers may be included. Flavours can be added to the composition.

Administration Period

The start of the administration period typically coincides with the beginning of the weaning period, i.e., when the first non-milk food is introduced. Alternatively, the B. lactis NCC2818 administration may begin shortly before this time, for example, one or two weeks before the introduction of the first non milk food. It may also occur shortly after the introduction of the first non-milk food. However the positive effects are thought to be greatest if the intervention with the probiotic coincides with the first introduction of novel foods or before this point.

For human infants, the age at which weaning starts may depend on the culture into which the infant is born, as weaning takes place at different ages according to different cultures. Often, weaning starts when the infant is between about 3 to 7 months old. Thus, in that case, the probiotic administration would begin when weaning starts, i.e. when the infant is between about 3 to 7 months old, or 1-4 weeks before this point.

The administration may even start earlier, for example 3, 4, 5, 6, 7, 8, 9 or 10 weeks before weaning starts.

The period of administration of the probiotics can be continuous, for example, every day up until the infant is at least 12 months old. Continuous administration is preferred for a more sustained effect. However, it is speculated that a discontinuous pattern (for example, daily administration during one week per month, or during alternate weeks) can induce beneficial effects on the infant.

The duration of the probiotic administration may vary which differs according to the infant and to the culture into which he is born. Positive effects are expected with even a short duration of administration, for example for one, two or three months, if administration begins at the same time as weaning or slightly earlier. A longer duration will provide a positive effect in the young mammal for a longer time. Typically, the probiotic administration is continued until the infant is at least 12 months old. The administration may be continued up until the infant is 18 months, or 24 months or even up to 3 years old. Infants generally continue to regularly encounter new foods up until the age of 4 years.

Preferably, the administration to the infant is by daily intake or intake is every other day, the probiotic being taken once or twice a day.

Effect of the Probiotic Administration

B. lactis NCC2818 administered to infants during the weaning period improves tolerance to newly introduced foods. This has been demonstrated in a set of experiments, using a piglet weaning animal model, as detailed in Example 1. A piglet model was chosen by the inventors to investigate the impact of B. lactis NCC2818 at weaning, because piglets are more comparable to humans than are rodents in their development at birth and postnatally. Additionally, a recent comparison of 147 genotypic, phenotypic and functional parameters in mice, pigs and humans has shown that 80% of these parameters were more akin between pigs and human than between mice and humans (Wernersson R, Schierup M H, Jorgensen F G, et al., 2005, Pigs in sequence space: A 0.66× coverage pig genome survey based on shotgun sequencing. BMC Genomics, 6:70).

The results presented herein clearly demonstrate that administration of B. lactis NCC2818 to piglets at weaning can have marked effects on the structure and function of the gut associated mucosal immune system.

In one embodiment of the invention, the transient increase of systemic IgGs specific to a newly introduced protein, which is normally observed during weaning, is enhanced. The increase occurs more quickly and to a greater extent, when weaning is accompanied by administration of B. lactis NCC2818.

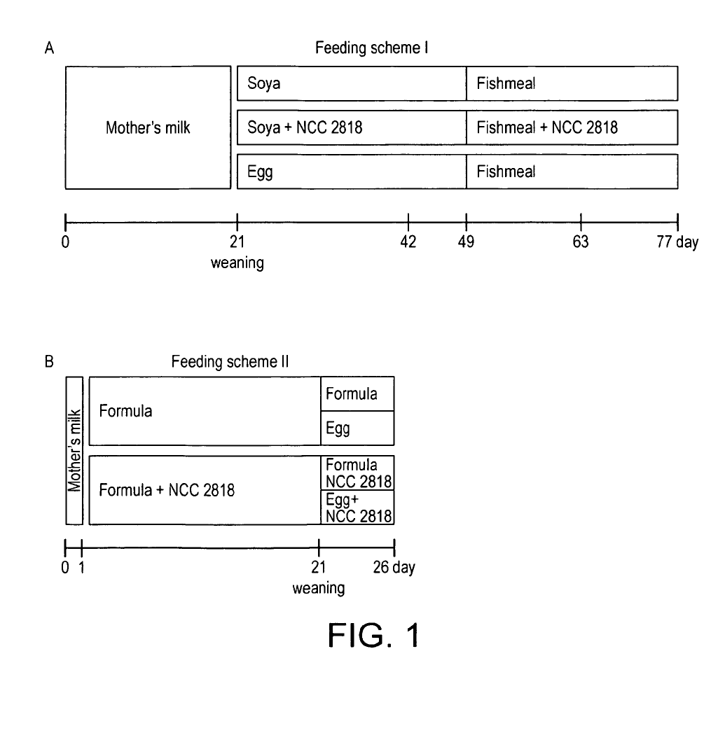

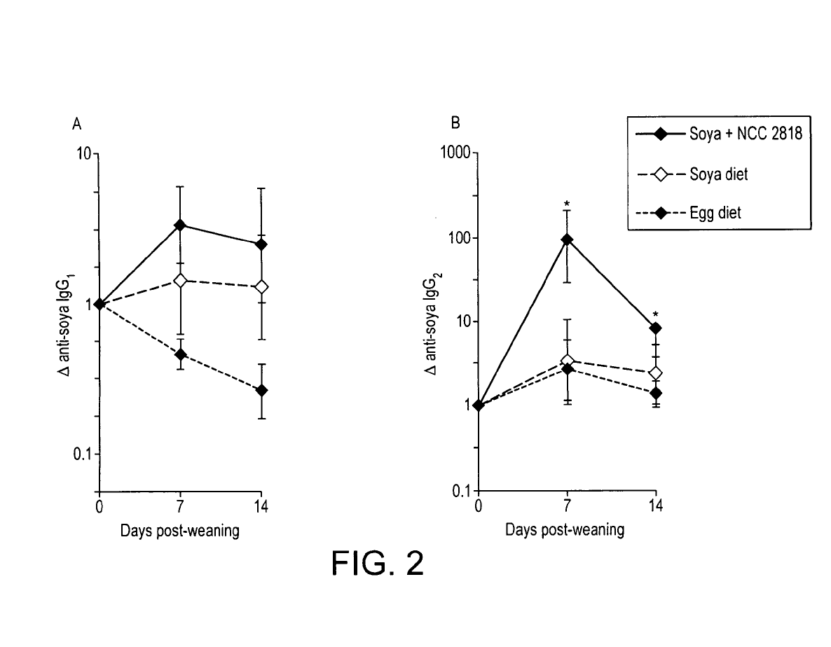

Thus, in Example 1, piglets, fed according to the Feeding Scheme 1 in FIG. 1A, were weaned from mother's milk at 3 weeks, onto either a soya diet, a soya diet supplemented with B. lactis NCC2818 mixed into the formula at a concentration of 4.2×10⁶ cfu/ml (approximately 2×10⁹ cfu/kg metabolic wt/day), or onto an egg diet. The levels of soya specific IgG1 and IgG2 in the serum of the animal in each group were measured at 0, 7 and 14 days post-weaning (see FIG. 2). This corresponds to 21, 28 and 35 days post birth in FIG. 1A. It was observed that feeding piglets at weaning with protein, previously unknown to the immune system, results in a transient increase of specific IgG in the serum, one and two weeks after weaning. It was also observed that when supplemented with B. lactis NCC2818 soya-fed piglets show a significantly higher increase in serum soya-specific IgG₂ (p=0.03; FIG. 2B) and a tendency of higher increase in soya-specific IgG₁ (FIG. 2A).

Elevated serum IgG antibody responses to food proteins have been associated with decreased susceptibility to IgE-mediated allergic disease in humans and to postweaning diarrhoea in pigs (Li, D. F. et al., Interrelationship between Hypersensitivity to Soybean Proteins and Growth-Performance in Early-Weaned Pigs, Journal of Animal Science, 1991; 69:4062-4069 and Strait, R. T., et al. Ingested allergens must be absorbed systemically to induce systemic anaphylaxis, Journal of Allergy and Clinical Immunology; 127:982-989.e1.).

Thus, the higher transient increase in soya specific IgGs observed in the B. lactis NCC2818 supplemented piglets of Example 1 indicates that the administration of B. lactis NCC2818 during weaning accelerates and increases the level of adaptation of the piglets immune system to the newly introduced protein.

In another embodiment, the villus height of the young mammal increases when weaning is accompanied by administration of B. lactis NCC2818.

Villus height may be seen as an indicator of good health in infants. Villus atrophy is frequently seen in accompanying diseases of the gastrointestinal tract like celiac disease or virus infections (Cummins, A. et al., American Journal of Gastroenterology, 2011, 106, 145-50; and Boshuizen, et al.; Journal of Virology, 2003, 77 (24), 13005-16). It has also been shown in piglets that the acute impairment of the intestinal integrity at weaning is, among others, indicated by a decrease in villus length. On the contrary, the adaptation that follows this period is marked by an increase in villus length in the jejunum (Montagne, L. et al., British Journal of Nutrition, 2007, 97, 45-57). Thus, a greater villus height is associated with an intestine that is becoming adapted to new foods.

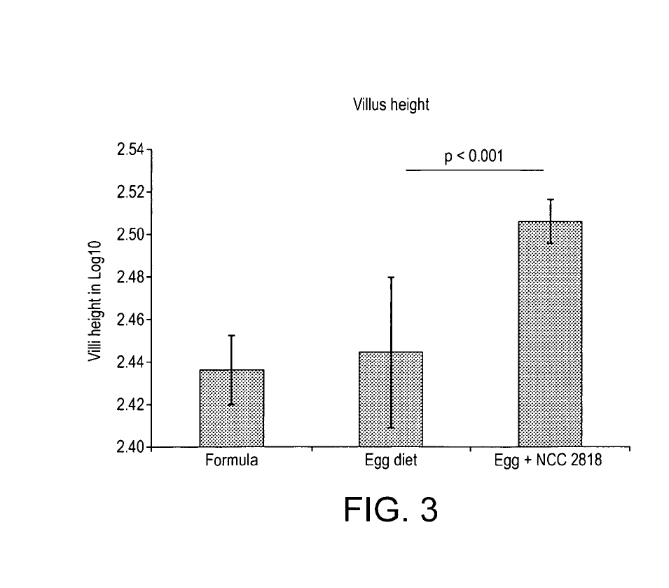

FIG. 3 shows the histomorphometry of the intestinal mucosa (distal small) of animals after following feeding scheme II in FIG. 1. Acute changes in mucosa morphology due to weaning occur between 2-5 days after weaning. Because the aim of the experiment was to demonstrate a beneficial impact of B. lactis NC2818 on the intestinal mucosa morphology, the experimental protocol of Feeding scheme II was adjusted accordingly. As it is believed that the probiotic needs a certain feeding duration to achieve an effect the feeding in these animals was started at 24 h of age. Thus, villus height (A) was measured for piglets fed from birth with or without B. lactis NCC2828 from 24h onwards. Pigs were either weaned onto solid food (Egg diet, Egg diet+NCC2818) at day 21 or kept on piglet formula (Formula).

Villus height was measured at day 25. Panel A demonstrates an increase in villus height in the group egg supplemented with B. lactis NCC2818 compared to the non-supplemented group. A sufficient villus height is generally regarded as one of the signs of a physiologically functional and well-developed intestinal mucosa. Safeguarding of villus height is generally regarded as protective. The increase of villus height by B. lactis NCC2818 can therefore be regarded as sign of mucosal protection.

In another embodiment, supplementing with B. lactis NCC2818 at weaning seems to promote a switch for certain immune processes in the mesenteric lymph nodes (MLN), from a less mature, IgM-dominated, antibody response to more mature IgA-dominated response.

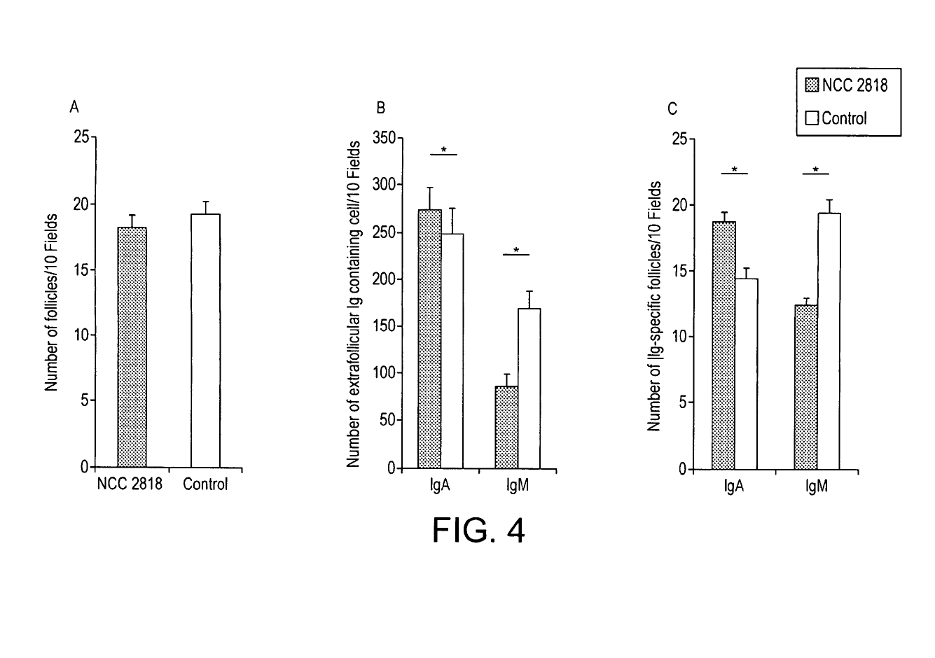

FIG. 4 show the fluorescence immunohistology of B-cell follicles in MLN of the animals of Example 1 Feeding Scheme I. Comparing the supplemented group (with B. lactis NCC2818) to the non-supplemented group, one observes that the total number of follicles in the lymph node is left unchanged (FIG. 4A). However, in the supplemented group there are significant decreases in the number of both IgM specific follicles and extrafollicular IgM producing cells (p<0.0001; FIGS. 4B, C). There are also significant increases in the number of IgA specific follicles (p=0.04 and p<0.0001 respectively; FIGS. 4B, C), compared to the non supplemented group.

These results are indicative of a move towards a more “mature” immune response to the newly introduced food protein in the animals supplemented with B. lactis NCC2818 during weaning. This more mature response can be viewed as an improvement of tolerance to newly introduced foods. The intestinal system of the young mammal is adapting faster to new foodstuffs. Thus, the inventors hypothesise that this faster adaptation would translate into a reduction of the vulnerable period associated with weaning. Thus, pathological conditions associated with weaning are prevented, or their severity reduced. Furthermore, the long-term effects of these conditions later in life are prevented and/or reduced.

Thus, administration of the probiotic according to the invention has a prophylactic effect on the young mammal, preventing mild discomfort or severe discomfort associated with pathological states that may result from the introduction to novel foods during the weaning period.

### EXAMPLES

### Example 1

### Piglet Model to Investigate the Impact of B. Lactis NCC2818 at Weaning

Two Experiments were Carried Out.

In the first experiment according to Feeding scheme I, (FIG. 1A) for the first three weeks of life, piglets were left suckling with their mothers. At week 3, animals were weaned on solid food with protein content based either on soya supplemented with B. lactis (NCC2818) or non-supplemented soya, or onto a non supplemented ovalbumin (OVA) diet. All animals were switched to a fishmeal diet at 7 weeks, with one group maintaining supplementation with B. lactis NCC2818. Animals were sacrificed at 11 weeks.

Levels of systemic soya specific IgGs were measured at 0, 7 and 14 days post weaning (FIG. 2), and levels of IgA, IgM and CD21 were examined in mesenteric lymph node (MLN) cells (FIG. 4) at sacrifice.

In the second experiment according to Feeding scheme II, (FIG. 1B) piglets were fed formula, which was either supplemented with B. lactis NCC2828 or not supplemented, from 24 h onwards. Pigs were either weaned onto solid food (Egg diet, Egg diet+NCC2818) at day 21, or kept on piglet formula (Formula). Villus height of samples of intestinal mucosa were measured at day 25, the day on which the pigs were sacrificed. The results are shown in FIG. 3.

The Experimental Details are Given Below.

Animal Model:

Animal housing and experimental procedures were all performed according to local ethical guidelines: all experiments were performed under a UK Home Office License and were approved by the local ethical review group. Seven outbred sows were artificially inseminated using semen from a single boar (supplied by Hermitage-Seaborough Ltd, North Tawton, Devon, UK). Sows were transported to the department of Clinical Veterinary Science six weeks prior to parturition and fed on a wheat-based diet (BOCM Pauls Ltd, Wherstead, UK).

Feeding Scheme I (FIG. 1A)

At 3 weeks of age, the piglets were weaned and litter-matched into three groups. At this point, one group received the Bifidobacterium animalis subsp. lactis (CNCM I-3446), otherwise known as B. lactis NCC2818, probiotic diet supplementation in the form of spray-dried culture mixed into the formula at a concentration of 4.2×10⁶ CFU/ml (approximately 2×10⁹ cfu/kg metabolic wt/day). The required quantity of feed supplemented with fresh probiotics was fed twice a day to the appropriate group until the experiment concluded when the pigs were 11 weeks old. The remaining two groups did not receive the probiotic supplement. Probiotic-fed and control animals were in different suites separated by a biosecurity barrier. The piglets receiving probiotics were weaned onto a soya based diet, while the piglets not receiving the probiotics were either weaned onto soya or ovalbumin (egg) diets. All diets were supplemented with appropriate levels of vitamins and minerals and were manufactured to order by Parnutt Foods Ltd (Sleaford, Lincolnshire, UK).

From 7 weeks old, all three groups were fed a fish-based diet, free of egg and soya, either with or without probiotic as appropriate.

All piglets were bled by venipuncture at 3, 4 and 5 weeks old for collection of serum. At 11 weeks old, piglets were sedated with azaperone and euthanized with an overdose of barbiturate. At post-mortem, heart-blood and tissues were recovered.

Feeding Scheme II (FIG. 1B)

At 1 day old, the piglets were separated from their mother and litter-matched into two groups. Then, up until day 21, one group was fed formula supplemented with Bifidobacterium animalis subsp. lactis (CNCM I-3446), otherwise known as B. lactis NCC2818, in the form of spray-dried culture mixed into the formula at a concentration of 4.2×10⁶ cfu/ml (approximately 2×10⁹ cfu/kg metabolic wt/day). The second group was fed formula without B. lactis NCC2818 supplementation, up until day 21. The required quantity of feed supplemented with fresh probiotics was fed twice a day to the supplemented group until the experiment concluded when the pigs were 25 days old.

When the piglets were three weeks old the B. lactis NCC2818 supplemented group were split into two groups and either weaned onto an egg diet supplemented B. lactis NCC2818 or not weaned at all. Similarly the non-supplemented group was split into two groups and either weaned onto an egg diet or not weaned at all. All diets were supplemented with appropriate levels of vitamins and minerals and were manufactured to order by Parnutt Foods Ltd. (Sleaford, Lincolnshire, UK). The diet was designed such that it contained 21% of egg protein.

At 25 days old, piglets were sedated with azaperone and euthanized with an overdose of barbiturate. At post-mortem, tissue was recovered.

Measurement of Antigen-Specific Immunoglobulin (FIG. 2)

Serum samples were taken from animals from Feeding Scheme I at 0, 7 and 14 days. The samples were analysed for anti-ovalbumin IgG₁ and IgG₂ antibodies by ELISA as described in detail in Bailey M, et al. Effects of infection with transmissible gastroenteritis virus on concomitant immune responses to dietary and injected antigens, Clin. Diagn. Lab. Immunol. 2004; 11:337-43. Briefly, 96 well microplates were coated with ovalbumin from chicken egg white (Sigma) before non-specific binding sites were blocked with 2% bovine serum albumin (BSA) (Sigma) in PBS-tween 20. After washing, serial dilutions of serum samples and reference standard were added to the plates. Reference standard was porcine serum obtained following hyperimmunisation with ovalbumin. Bound anti-soya IgG₁ and IgG₂ antibodies were detected using isotype-specific monoclonal antibodies followed by HRP-conjugated goat anti-mouse as above, and relative concentrations of antibody were determined by interpolation of samples onto the reference standards.

In order to compare changes in serum antibody generated by weaning in outbred animals, in which the starting levels differ, results are expressed as the ratio of antibody after manipulation to that before manipulation (the —fold change in antibody).

Immunohistology

Sample Collection

MLN tissue was removed shortly after death from each of the experimental piglets. Tissues were embedded in OCT (Tissue TEK, BDH, Lutterworth, Leicestershire, UK) and snap-frozen in isopentane, pre-cooled to approximately −70° C. in the vapour phase of liquid nitrogen. Samples were stored at −80° C. until sectioning. Serial, 5 μm sections of these tissues were cut using a Model OTF cryotome (Brights Instrument Company Ltd., Huntingdon. UK). Sections were air dried for 24 h then fixed by immersion in acetone for 15 min. Slides were allowed to dry before storage at −80° C.

Fluorescence Immunohistology and Analysis

For 2 colour fluorescence immunohistology, mouse anti-pig monoclonal antibodies (IgA and IgM, as for ELISA) were used to identify free and cell-bound IgA and IgM positive cells (FIG. 4). The conjugated secondary reagents used were: goat anti-mouse IgG₁ conjugated to FITC (Southern Biotechnology, AMS Biotechnology, Oxon, UK) and goat anti-mouse IgG_{2b} conjugated to TRITC (Southern Biotechnology). Tissue staining, image capture and automated image analysis were carried out as described by Inman et al, 2010, Inman, C. F., Rees, L. E. N., Barker E., Haverson, K., Stokes, C. R., Bailey, M., Validation of computer-assisted, pixel-based analysis of multiple-colour immunofluorescence histology, Journal of Immunological Methods, 2005; 302:156-167 with the exception that Fc receptor blocking was achieved using 10% goat serum in PBS.

Histomorphometry and Analysis

Samples were prepared as indicated above in sample collection and stained with hematoxylin and eosin stain and subsequently analysed with image capture and automated image analyse using Image software to detect villus length.

### Example 2

Starter Formula

| Nutrient | per 100 kcal | per litre |
|---|---|---|
| Energy (kcal) | 100 | 670 |
| Protein (g) | 1.83 | 12.3 |
| Fat (g) | 5.3 | 35.7 |
| Linoleic acid (g) | 0.79 | 5.3 |
| α-Linolenic acid (mg) | 101 | 675 |
| Lactose (g) | 11.2 | 74.7 |
| Prebiotic (100% GOS) (g) | 0.64 | 4.3 |
| Minerals (g) | 0.37 | 2.5 |
| Na (mg) | 23 | 150 |
| K (mg) | 89 | 590 |
| CI (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| I (μg) | 15 | 100 |
| Cu (mg) | 0.06 | 0.4 |
| Zn (mg) | 0.75 | 5 |

| | |
|---|---|
| B. Lactis NCC2818 | 2 × 10⁷ cfu/g |
| | of powder |

### Example 3

Growing Up Milk Compositions

| Nutrient | per 100 kcal | | | | | |
|---|---|---|---|---|---|---|
| Energy (kcal) | 100 | 100 | 100 | 100 | 100 | 100 |
| Protein (g) | 2.7 | 2.22 | 2.23 | 2.3 | 2.9 | 2.26 |
| Whey/Casein | 23/77 | 40/60 | 40/60 | 40/60 | 77/23 | 40/60 |
| CHO (g) | 12.2 | 13.5 | 13.1 | 13.0 | 11.9 | 13.9 |
| Lactose (g) | 5.05 | 6.7 | 6.1 | 4.9 | 4.42 | 5.33 |
| Maltodextrine | 4.99 | 5.8 | 5.5 | 4.9 | 2.31 | 2.35 |
| (g) | | | | | | |
| Starch (g) | | 1.0 | 1.0 | 2.9 | 2.29 | 3.17 |
| Sucrose (g) | 1.93 | | | | 2.66 | 2.41 |
| Fat (g) | 4.5 | 4.14 | 4.31 | 4.3 | 4.53 | 3.93 |
| Prebiotics (g) | | 0.58 | 0.58 | 0.52 | | 0.49 |

| | |
|---|---|
| B. Lactis | 2 × 10⁷ cfu/g of powder |
| NCC2818 | |

Further supporting evidence for the present invention is to be found in the paper “Weaning diet induces sustained metabolic phenotype shift in the pig and influences host response to Bifidobacterium lactis NCC2818C” (Merrifield and M. Lewis et al., 2012, Gut doi:10.1136/gutjnl-2011-301656), herewith incorporated by reference. Particular reference to FIG. 3 panel A of Merrifield and M. Lewis eta/is made. The data shown in Merrifield and M. Lewis et al provide evidence that a probiotic, specifically Bifidobacterium animalis subsp. lactis, has an effect on immune adaptation when administered to healthy young mammals during the weaning period.

1. A method for improving tolerance to newly introduced foods during the weaning period of healthy young mammals comprising administering to the young mammal a probiotic.

2. The method according to claim 1 wherein the transient increase in the humoral immune response, upon exposure to newly introduced foods, occurs more rapidly and/or to a greater extent, compared to that occurring in young mammals not receiving the probiotic.

3. The method according to claim 1 wherein, during weaning, the height and/or area of the intestinal mucosal villi is increased by more than 15% compared to that of young mammals not receiving the probiotic.

4. The method according to claim 1 wherein the probiotic is a Bifidobacterium animalis.

5. The method according to claim 1 wherein the probiotic is a Bifidobacterium animalis subsp. lactis (B. lactis).

6. The method of claim 1 wherein the probiotic is strain B. lactis NCC2818.

7. The method of claim 1 wherein the probiotic is administered as a daily dose of from 1×10² to 1×10¹¹ cfu (cfu=colony forming unit).

8. The method of claim 1 wherein the probiotic is administered to healthy young humans aged between about 3 months and about 24 months.

9. The method of claim 1 wherein the probiotic is administered in a form selected from the group consisting of its pure form, diluted in water, and in a composition suitable for administration to young mammals.

10. The method of claim 1 wherein the probiotic is administered in combination with an additional probiotic.

11. The method according to claim 10 wherein the additional probiotic is selected from the group consisting of Bifidobacterium longum BB536 (ATCC BAA-999); Lactobacillus rhamnosus (CGMCC 1.3724); Lactobacilus reuteri (DSM 17938) and mixtures thereof.

12. The method according to claim 1 wherein the probiotic is administered in a composition wherein the composition comprises further ingredients selected from the group consisting of inulin, fructooligosaccharide (FOS), short-chain fructooligosaccharide (short chain FOS), galactooligosaccharide (GOS), xylooligosaccharide (XOS), arabinoxylan-oligosaccharides (AXOS), glanglioside, partially hydrolysed guar gum, acacia gum, soybean-gum, Lactowolfberry, wolfberry extracts and mixtures thereof.

13. The method of claim 1 wherein the probiotic has been inactivated such as to render it non-replicating.

14. The method according to claim 1 wherein the probiotic is administered in a form selected from the group consisting of an infant formula, follow-on formula, growing-up milk, cereal or yoghurt, a baby meal, pudding or cheese, a dairy or fruit drink, a smoothy, a snack, biscuit and other bakery item.

15. A method to accelerate adaptation to newly introduced foods in a healthy young mammal during the weaning period comprising administering a composition comprising a probiotic to the healthy young mammal.

16. The method according to claim 15 wherein the probiotic or mixture of probiotics is:
administered to the young mammal as a daily dose of a probiotic when weaning begins or shortly before weaning; and
continued to be administered daily for a period of at least 4 weeks after the weaning period begins.

17. The method according to claim 16, wherein the probiotic is B. lactis NCC2818.

18. The method according to claim 17, wherein the daily dose is about 1×10⁶ to about 1×10⁹ colony forming units (cfu) of B. lactis NCC2818.
